(19) United States
(12) Patent Application Publication
(10) Pub. No.: US 2001/0041864 A1
(43) Pub. Date: Nov. 15, 2001

(54) Arterial perfusion cannula and method for providing cardiopulmonary bypass

(76) Inventor: William P. Sweezer, Lafayette, CA (US)

Correspondence Address: AUDLEY A. CIAMPORCERO JR., JOHNSON & JOHNSON, ONE JOHNSON & JOHNSON PLAZA, NEW BRUNSWICK, NJ, 08933-7003, US

(21) Appl. No.: 09/908,187

(22) Filed: Jul. 18, 2001

Related U.S. Application Data
(62) Division of application No. 09/173,248, filed on Oct. 15, 1998.

(51) Int. Cl. A61M 37/00; A61M 31/00

(52) U.S. Cl. 604/101.03; 604/509; 604/6.16

(57) ABSTRACT

An arterial perfusion cannula and method for achieving cardiopulmonary bypass during heart surgery. The arterial perfusion cannula is inserted peripherally into a preselected arterial vessel and advanced within the vessel and positioned in the ascending aorta cephalid of the junction of the coronary arteries with the aortic root. The arterial cannula has an arterial return lumen for returning oxygenated blood from to the patient from a cardiopulmonary bypass system. The arterial perfusion cannula further has a cardioplegia/venting lumen for the passage of cardioplegia solution to arrest the heart or for the evacuation of blood from the aortic root. An expandable occlusion member is provided for internal occlusion of the ascending aorta between the coronary arteries and the brachiocephalic artery.

### FIELD OF THE INVENTION

This invention relates to catheters for use in providing cardiopulmonary bypass support and isolation of the heart during the performance of heart surgery. More specifically, the invention relates to catheters for aortic occlusion, aortic root cardioplegia delivery, aortic root venting, and left ventricular decompression without the necessity for a conventional open chest operation.

### BACKGROUND OF THE INVENTION

Each year cardiopulmonary bypass permits over 500,000 patients worldwide with disabling heart disease to undergo therapeutic cardiac operations. The essential goals of cardiopulmonary bypass for heart surgery are to provide life-support functions, a motionless, decompressed heart, and a dry, bloodless field of view for the surgeon.

In a basic heart-lung life-support system oxygen-poor blood is diverted from the venous circulation of the patient and is transported to a cardiopulmonary bypass system (or heart-lung machine) where reoxygenation occurs, carbon dioxide is discarded and heat regulation (warming or cooling) is accomplished. This processed blood is then returned (perfused) into the patient's arterial circulation for distribution throughout the entire body to nourish and maintain viability of the vital organs. Although current venous diversion and arterial perfusion methods can be combined with other measures to effectively isolate the heart for cardiac surgery, they are associated with disadvantages and limitations which contribute significantly to patient morbidity, mortality, and health care costs. It is thus desirable to develop improved cardiopulmonary bypass devices and methods that are safer, less traumatic, and more cost effective.

In order to perform complex, delicate surgical procedures on the heart, i.e., coronary artery bypass and valve operations, it is desirable to establish a resting, non-beating (flaccid) non-distended state. This condition, along with a dry, bloodless field, is ideal for safe manipulation and suturing of cardiac structures, and furthermore, contributes to decreased metabolic cardiac energy demands while promoting preservation of cellular functions. In the prior art this non-beating state was accomplished by delivery of a cardioplegia (heart paralyzing) solution to the coronary circulation to stop the heart by one or a combination of two general methods: (1) Antegrade (cardioplegia infusion is initiated at the arterial end of the coronary circulation via the origins of the coronary arteries, i.e., ostia, in the aortic root and flows towards the capillaries within the heart muscle; (2) retrograde (cardioplegia infusion is directed into the venous circulation via a coronary sinus and flows backwards into the capillary circulation of the heart muscle). It is at the capillary level where the cardioplegia solution interacts with the cardiac muscle cells, resulting in its desired effects.

Most conventional antegrade cardioplegic techniques for heart surgery require an external occlusive vascular clamp to be applied to the ascending aorta to prevent arterialized blood from the cardiopulmonary bypass pump from reaching the coronary arteries, proximal ascending aorta, and aortic valve areas while at the same time maintaining arterial perfusion to all points distal (downstream) to the clamp. This isolation maneuver then allowed infusion of cardioplegia solution either directly into the coronary openings (ostia) via catheters, (cannulas) whose tips were inserted into the ostia or indirectly via a catheter (cannula) inserted into the isolated segment of the ascending aorta adjacent to the coronary ostia. Surgical trauma to the aorta resulted from the aortic puncture wounds or major aortic incisions that had to be made to use these techniques, both of which were dependent on major sternotomy or thoracotomy for exposure. The use of the surgical clamp to squeeze the opposing aortic walls together also has major disadvantages. For instance, a major invasive surgical incision (sternotomy or thoracotomy) is required to reach the aorta in order to apply the clamp. By the compressing or squeezing action of the clamp, fragments of cholesterol or calcium in the aortic wall may break away and embolize to the vital organs downstream. In cases of very severe calcification of the ascending aorta, it is not feasible to apply an external clamp because the compressibility of the aorta has been lost. Surgeons must then resort to less optimal, more complex methods of bypass support, myocardial protection and heart isolation which further increases the likelihood of post-operative complications. There are situations where the surgeon cannot proceed with the operation and it is terminated with the patient losing the opportunity for definitive therapeutic treatment of his disabling heart disease. Most conventional retrograde prior art cardioplegia delivery methods also are dependent upon major invasive chest operations as well as direct trauma to the atrium for their use. Again, the patient is being subjected to increased risks of bleeding and direct cardiac trauma.

Prior art methods of controlling distention (decompression or venting) and improving visibility of the heart during heart surgery included: (1) insertion of a catheter via the left atrium or a pulmonary vein which was then directed across the mitral valve so that its openings at the tip were positioned within the left ventricular chamber for suction evacuation (also called venting) of blood; (2) inserting a catheter directly into the apex of the left ventricular muscle so that its openings at the tip were positioned within the left ventricular chamber for suction evacuation (venting) of blood; and (3) the prior art catheter placed in the isolated segment of the ascending aorta for antegrade cardioplegia delivery could alternatively be switched to a suction source to accomplish aortic root venting (decompression) but not left ventricular decompression (venting). All of these methods have the disadvantages of requiring major stemotomy or thoracotomy and are associated with direct cardiac and aortic trauma.

When surgeons are required to perform repeat open heart surgery (known as “redo” operations) in someone whose chest has previously been entered via a major sternotomy or thoracotomy, extensive adhesions are usually encountered which obliterate the natural relationship and appearance of anatomic structures. This distortion further increases the risks of injury and massive fatal hemorrhage during the process of exposing, isolating and preparing structures for catheter insertions (arterial, venous, cardioplegia, left ventricular vent) and therapeutic repair.

Major invasive chest incisions are often associated with a higher incidence of morbidity including, but not limited to, intraoperative and post-operative bleeding, resulting in the likelihood of increased blood transfusion requirements, returns to surgery for re-exploration to control hemorrhage, longer healing and recovery times, pulmonary complications (such as lung collapse and pneumonia), catastrophic wound infection (mediastinitis), extensive scarring and adhesions, mechanical wound instability and disruption (dehiscence), chronic incisional pain, peripheral nerve and musculoskeletal dysfunction syndromes. Developing a system with features that avoid surgical maneuvers, instrumentation and devices known to be associated with increased morbidity and mortality is desirable. Such improvements have the likelihood of resulting in a favorable impact on patient care, quality of life, and health care costs.

### SUMMARY OF THE INVENTION

In accordance with the present invention, a multichannel catheter is provided for providing bypass support. The multichannel catheter has a first lumen or channel extending substantially the length of the catheter with the first channel comprising a major portion of an available channel volume of the catheter. The first lumen being defined by the wall of the catheter and being closed at its distal end. The multichannel catheter also has a second lumen or channel extending substantially the length of the catheter parallel to said first channel but independent thereof. The second lumen is integrated into the wall of the first channel and is open at its distal end. The catheter also has a third lumen or channel extending substantially the length parallel to the first and second lumens but independent thereof. The third lumen, in combination with the second lumen, comprises a minor portion of the available channel volume of the catheter and is integrated into the wall of the first lumen or channel. The third lumen also has an opening and is spaced from the second channel. The catheter also has a plurality of first outlets in the wall of the catheter near the distal end of said catheter which communicate only with said first channel. An inflatable means is integrated into the distal end of the catheter between the first outlets and the second lumen opening. The opening of said third lumen is in fluid communication with the interior of the inflatable means for inflating the inflatable means. The catheter is preferably of a size suitable for insertion into a blood vessel of mammal and preferably has a length sufficient to allow insertion into a femoral artery and positioning such that the distal end of the catheter is located in the ascending aorta and the outlets are positioned substantially adjacent the great arteries.

In a preferred embodiment, the first lumen preferably comprises at least about ninety three percent of the available channel volume of the catheter and the third lumen, in combination with the second lumen, comprises not more than about seven percent of the available channel volume of the catheter. In another preferred embodiment, the first lumen comprises at least about seventy percent of the available channel volume of the catheter and the third channel, in combination with the second channel, comprises not more than about thirty percent of the available channel volume of the catheter.

In another aspect of the invention, the outlets coupled to the first lumen have an outflow capacity which exceeds the inflow capacity into the first lumen. Furthermore, the plurality of the outlets are preferably elongate with the length of each elongate outlet being parallel to the length of the catheter. The catheter may be formed using any suitable method including an extrusion technique such as an extrusion molding. The catheter is preferably formed with all of the lumens being integrally formed by a single catheter wall. The catheter preferably includes markers positioned near the proximal end of the catheter to mark the distance from the distal end of the catheter under fluoroscopic visualization.

The present invention is also directed to a process of preparing a multichannel catheter that is of a size suitable for insertion into a blood vessel of a mammal. The catheter may be formed in any manner and is preferably extrusion molded. A first lumen or channel is formed to extend substantially the length of the catheter and comprises a major portion of the available channel volume of the catheter. A second lumen or channel extends substantially the length of the catheter parallel to the first lumen but independent thereof. The first and second lumens are both preferably integrated into the same structure so that they share the same catheter wall. A third lumen or channel extends substantially the length of the catheter parallel to the first and second lumens but independent thereof. The third lumen comprises, in combination with the second channel, not more than a minor portion of the available channel volume of the catheter. The third lumen is integrated into the wall of the first channel and spaced from said second channel. In a preferred method of the invention, the first channel comprises at least about ninety three percent of the available channel volume. In yet another preferred method, the first lumen comprises at least about seventy percent of the available channel volume while the second and third lumens comprise no more than about thirty percent of the available channel volume.

A plurality of outlets are formed in the wall of the catheter near the distal end which communicate only with the first lumen. An inflatable means, such as a balloon, is integrated into the distal end of the catheter and positioned distal to the first lumen outlets. The interior of the inflatable means is in fluid communication with the third lumen through an opening in the wall of the catheter. The distal end of the first channel is preferably closed so that oxygenated blood passes through the outlets.

In yet another method according to the present invention, a process for providing oxygen-rich blood to a patient's arterial circulation while providing a biologically active fluid to the heart of the subject is provided. A catheter is positioned so that the distal end is in the patient's aorta. The catheter has a first lumen or channel extending substantially the length of the catheter. The first lumen comprises a major portion of the available channel volume of the catheter and is closed at the distal end. In other preferred methods, the first lumen comprises at least seventy percent or at least ninety three percent of the available channel volume of the catheter. A second lumen or channel and a third lumen or channel both extend substantially the length of the catheter parallel to said first lumen but independent thereof. The third lumen comprising, in combination with the second lumen, a minor portion of the available channel volume of the catheter. In other preferred methods, the third lumen, together with the second lumen, comprises no more than about thirty percent while the first lumen comprises at least about seventy percent of the available channel volume. The first, second and third lumens are all integrated into the wall of the catheter.

A plurality of outlets or openings are formed near the distal end of the catheter which are in communication only with the first lumen. At least one opening is formed at the distal end of the catheter which communicates with the second lumen. An inflatable means is integrated into the distal end of the catheter between the first lumen outlets and the second lumen opening. The inflatable means communicates with the third lumen through an opening in the wall of the catheter.

A source of oxygen-rich blood is coupled to the proximal end of the first lumen and a source of biologically active fluid, such as cardioplegic fluid, is coupled to the proximal end of said second lumen. A source of fluid for inflating the inflatable means is provided at the proximal end of said third lumen.

The catheter is positioned within the subject's blood circulatory system such that the distal end of the catheter is positioned in the ascending aorta so that the first channel openings are located upstream of the inflatable means. The inflatable means is located on the cephalid side of the aortic valve and the distal end of the second lumen is located downstream of the inflatable means and proximate the aortic valve. The inflatable means is optionally inflated to block the flow of blood to the heart. The biologically active fluid is pumped to the heart and the oxygen-rich blood is pumped through the first lumen and out the first lumen outlets at a rate sufficient to maintain the subject's metabolism and perfusion. At this time, cardiovascular or cardiac surgery may be performed as needed. Circulatory support is, of course, maintained for the subject as needed.

In yet another aspect of the present invention, a single, multichannel catheter useful for extracorporeal circulation of blood to a patient undergoing cardiovascular surgery is provided. The catheter has at least three independent lumens or channels and an expandable balloon at one end of the catheter. A first, largest lumen is of a size to allow delivery of an amount of blood to the patient that is sufficient to support the patient metabolism and perfusion throughout the surgery. A second lumen, smaller than the first lumen, is preferably integrated into the wall of the first lumen. The second lumen is suitable for delivering cardioplegia solution to the heart and venting the left heart. A third lumen, which is also smaller than the first lumen, is also integrated into the wall of the first lumen. The third lumen is suitable for delivery of a fluid to the balloon for its expansion when positioned in the ascending aorta to occlude the flow of blood.

In a preferred embodiment of the multichannel catheter, the catheter has a length sufficient to be inserted throughout the femoral artery and positioned so that the balloon is positioned in the ascending aorta. Blood is delivered to the patient through openings in the wall of the first lumen that are upstream of the balloon. Cardioplegia solution is delivered and the left heart is vented through an opening in the second lumen that is downstream of the balloon.

In yet another method in accordance with the present invention, a method for performing cardiovascular surgery on a patient using a cardiopulmonary machine for extracorporeal circulation of blood is provided. The method utilizes a single, multichannel catheter for the extracorporeal circulation. The multichannel catheter includes at least three independent lumens or channels and an expandable balloon at the distal end of the catheter. A first, largest lumen or channel is of a size to allow delivery of an amount of blood to the patient that is sufficient to support the patient metabolism and perfusion throughout the surgery. A second lumen, smaller than the first lumen, is integrated into the wall of the first lumen. The second lumen is suitable for delivering cardioplegia solution to the heart and venting the left heart. A third lumen, also smaller than the first lumen, is also integrated into the wall of the first lumen. The third lumen is suitable for delivery of a fluid to the balloon for its expansion when positioned in the ascending aorta to occlude the flow of blood. Blood is delivered to the patient through the outlets in the wall of the first lumen that are upstream of the balloon. Cardioplegia solution is delivered through the second lumen opening that is downstream of the balloon. Once the patient is maintained by bypass support, surgery, such as open-chest surgery or minimally invasive cardiac surgery, may be performed. The catheter is preferably introduced into the patient's aorta or one of the great arteries and positioned so that the balloon is located in the ascending aorta to occlude the flow of blood to the heart.

These and other features and advantages will become appreciated as the same become better understood with reference to the following specification, claims and drawings.

### BRIEF DESCRIPTION OF THE DRAWINGS

FIG. 1 is a schematic drawing illustrating an arterial perfusion cannula according to the invention after introduction into a patient's vascular system.

FIG. 2 is a side cross-sectional view of a distal portion of the arterial perfusion cannula of FIG. 1.

FIG. 3 is a transverse cross-section of the arterial perfusion cannula of FIG. 2.

FIG. 4 is a side elevational view of an arterial perfusion cannula according to the invention in a further embodiment thereof.

FIG. 5 is a partial side elevational view of an arterial perfusion cannula according to the invention in another embodiment thereof.

FIG. 6 is a schematic drawing illustrating a further embodiment of an arterial perfusion cannula according to the invention in place in a patient's vascular system.

FIG. 7 is a schematic drawing of another arterial perfusion cannula according to invention.

### DETAILED DESCRIPTION OF SPECIFIC EMBODIMENTS

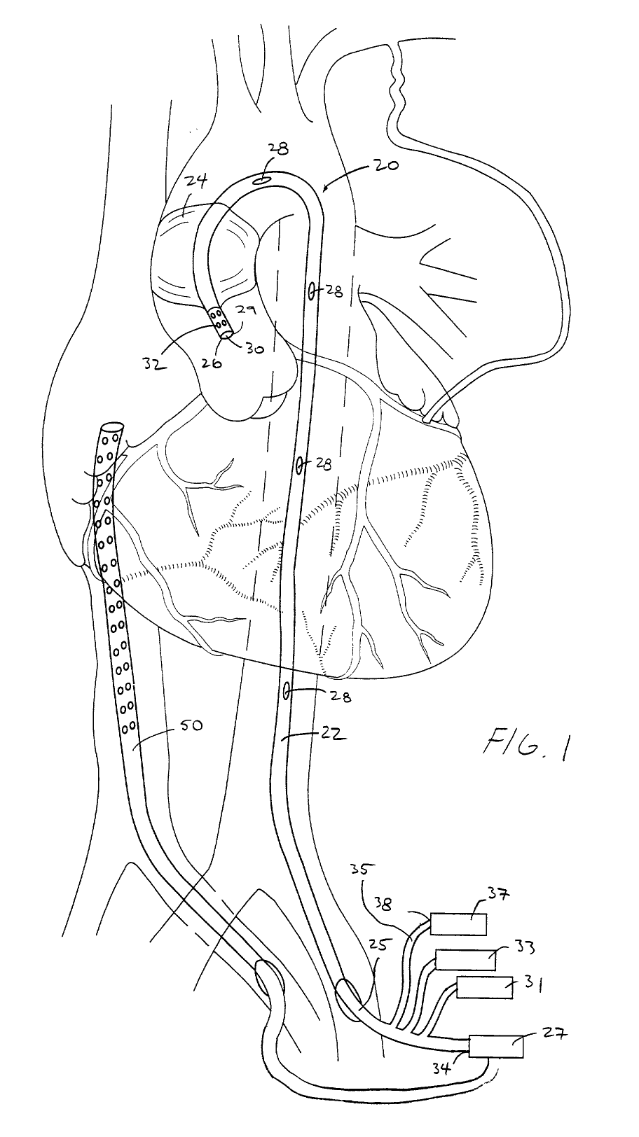

FIG. 1 illustrates a first embodiment of the arterial perfusion cannula of the invention as utilized in a patient's vascular system. Arterial perfusion cannula 20 has an elongated flexible shaft 22 suitable for placement into a peripheral artery such as the femoral or subclavian arteries (femoral arterial placement illustrated in FIG. 1), or for direct placement into the descending or ascending aorta. An occlusion member 24, which preferably comprises an inflatable balloon, is mounted to shaft 22 near its distal end 26. Occlusion member 24 is expandable to a size and shape suitable to occlude the ascending aorta downstream from the coronary arteries and upstream of the brachiocephalic artery, without blocking fluid flow into any of these arteries.

Referring to FIGS. 1-3, the shaft 22 has plurality of arterial outlets 28 positioned along its length proximally of occlusion member 24, each of which is in communication with an arterial return lumen 25 for return of oxygenated blood to the aorta from a cardiopulmonary bypass system 27. The cardiopulmonary bypass system 27 may be any suitable system and may include, for example, a pump and a blood oxygenation means fluidly connected to the pump. The outlets 28 are configured to provide adequate inflow of oxygenated blood to perfuse the patient during full cardiopulmonary bypass, which usually requires blood flow of at least about 4 liters/min. at pressures not exceeding about 300 mmHg. Preferably, arterial outlets 28 are positioned at spaced-apart locations along shaft 22 from a point immediately proximal to occlusion member 24 positioned in the ascending aorta, to a middle region of shaft 22 positioned in the descending thoracic aorta. The number and size of the outlets 28 are preferably selected so that the outflow capacity of the plurality of openings communicating with said first channel exceeds the inflow capacity into the first channel. As shown in FIGS. 1 and 2, the overall area of the outlets 28 exceed the size the arterial return lumen 25 thereby providing greater outflow capacity than the inflow capacity of the arterial return lumen 25. As shown in FIG. 1, the length of the catheter is sufficient to allow insertion into a femoral artery and positioning such that the distal end of the catheter is located in the ascending aorta with at least some of the outlets 28 positioned substantially adjacent the great arteries.

Shaft 22 further includes a distal opening 30 at its distal end 26 in communication with a lumen 29. A valve (not shown) is used to couple the lumen 29 to either a source of cardioplegic fluid 31 or a suction source 33 for venting blood. Distal opening 30 is configured to provide infusion of cardioplegic fluid from the source of cardioplegic fluid 31 into the ascending aorta distally of occlusion member 24 for purposes of stopping the heart during surgery. Additionally, blood and other fluids may be aspirated from the ascending aorta through distal opening 30 using the suction source 33 for venting the heart and aorta and maintaining a blood-free field. Shaft 22 may optionally include one or more side holes 32 near distal end 26 in communication with the lumen 29 to enhance venting and infusion of cardioplegic fluid.

At the proximal end of shaft 22 an arterial inlet 34 is in communication with the arterial return lumen 25 and is configured for connection to the outlet of the cardiopulmonary bypass system 27 for return of oxygenated blood to the patient. When the occlusion member 24 is an inflatable balloon, a balloon inlet 38 is in communication with an inflation lumen 35 coupled to a source of inflation fluid 37 which may be a syringe or other inflation device to allow delivery of an inflation fluid into occlusion member 24.

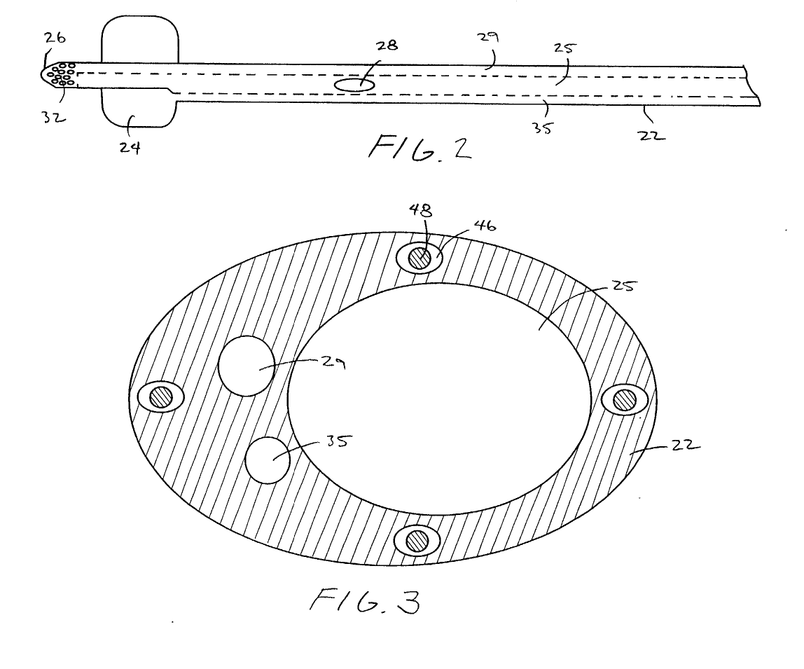

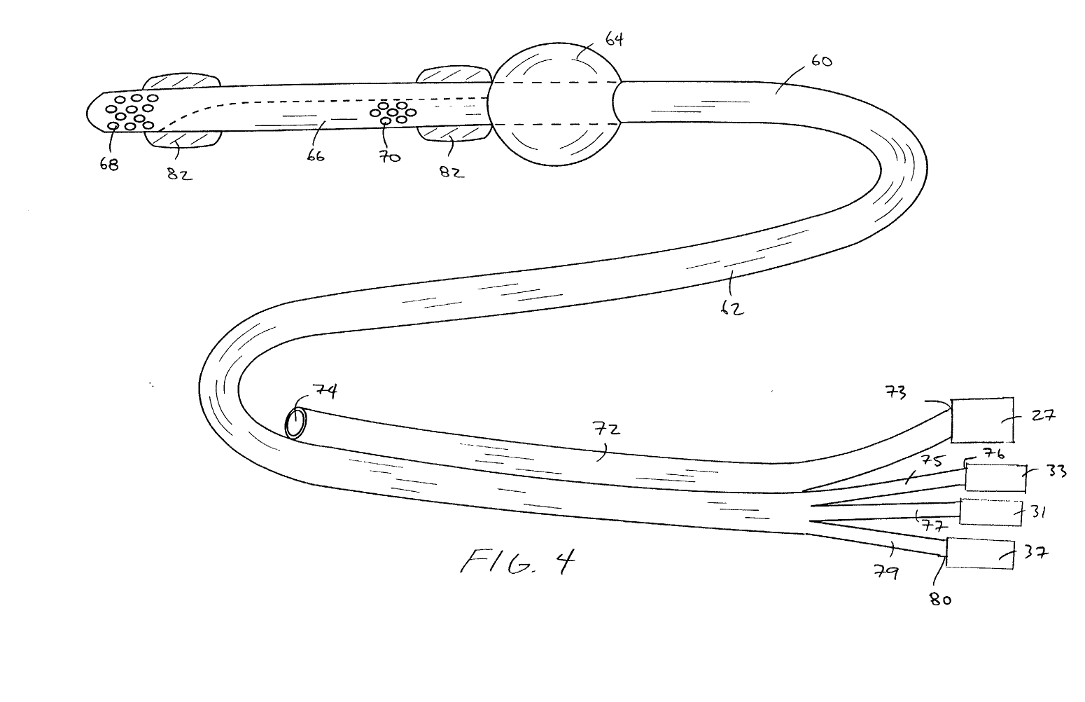

FIGS. 2 and 3 illustrate the arterial perfusion cannula 20 in cross-section. Shaft 22 includes the arterial return lumen 25, the cardioplegia/venting lumen 29 and the inflation lumen 35 integrated into the wall of arterial return lumen 25. Arterial return lumen 25 communicates with arterial outlets 28, which preferably are oval or other elongate shape having a longer axis generally parallel to the longitudinal axis of shaft 22. As shown in FIG. 1, a majority of the plurality of outlets 28 are elongate with the length of each elongate opening being parallel to the length of the catheter. The arterial return lumen 25 is preferably closed at the distal end as shown in FIG. 3. Internal walls separating the lumens 25, 29, 35 are depicted by the interrupted lines in FIG. 3.

Shaft 22 may optionally include wire channels 46 through which steering wires 48 may be slidably positioned. Steering wires 48 are fastened to the distal end 26 of shaft 22 and facilitate deflection or steering of distal end 26 by selective tensioning of the steering wires using a suitable actuation mechanism (not shown) at the proximal end of the arterial perfusion cannula. Such steerability facilitates manipulation of the arterial perfusion cannula through the tortuous vascular system and around the aortic arch into the ascending aorta.

Arterial return lumen 25 is dimensioned to provide sufficient blood flow to support the patient during full cardiopulmonary bypass, preferably having a cross-sectional area (or volume) which comprises a major portion of the total cross-sectional area (or volume) available for lumens in shaft 22, usually at least about 60% of the available lumen cross-sectional area (or volume), and preferably at least about 70% of the available lumen cross-sectional area (or volume) in shaft 22. For example in the embodiment illustrated in FIG. 3, arterial return lumen 25 has a cross-sectional area equal to about 93% of the total combined cross-sectional areas of the arterial return lumen 25, cardioplegia/venting lumen 29, and inflation lumen 35, or about 89% of the combined cross-sectional areas of all three lumens plus steering wire channels 46, described below. Stated another way, arterial return lumen 25 of FIG. 3 has a cross-sectional area of at least about 42% of the cross-sectional area of shaft 22 if steering channels 46 and steering wires 48 (described below) are utilized, or, if steering channels 46 and steering wires 48 are not utilized, arterial return lumen 25 has a cross-sectional area of at least about 60% of the cross-sectional area of shaft 22. Of course, the amount of the shaft cross-section available for the lumens is slightly less than the total shaft cross-section to allow for lumen wall thickness, so that arterial return 25 is a slightly greater percentage of the total available lumen area, e.g., at least about 65%-70% of the available lumen cross-sectional area (or volume).

Shaft 22 is constructed of a flexible biocompatible polymer such as silicon rubber, polyurethane, polyvinyl chloride, polyurethane, ethylene, nylon or other suitable catheter material. Shaft may be formed in any suitable manner such as an extrusion molding or other extrusion technique. The shaft 22 is formed so that all of the lumens 25, 29, 35 and channels 46 are formed and integrated into a single wall 49 of the shaft 22. If an inflatable balloon is utilized as occlusion member 24, it may be constructed of any of a number of well-known materials, including silicon rubber, polyurethane, latex, nylon, polyamide, and polyethylene.

In use, arterial perfusion cannula 20 is inserted into a peripheral artery such as the femoral artery and advanced into the aorta, around the aortic arch and into the ascending aorta. Arterial inlet 34 is connected to the outlet of the cardiopulmonary bypass system 27 to receive oxygenated blood therefrom. A venous cannula 50 is inserted into a peripheral vein such as a femoral vein and includes a plurality of blood inlets 52 for withdrawing blood from the patient. The cannula 50 is inserted into the peripheral vein (such as the femoral vein) so that the distal end is adjacent the vena cava regions of the heart. The proximal end of the cannula 50 is attached to the cardiopulmonary bypass system 27 which may include a cardiopulmonary machine and a pump with the cardiopulmonary machine having a blood oxygenation means fluidly connected to the pump. The cannula 50 is used to remove oxygen depleted blood from the vena cavae regions by applying a negative pressure using the cardiopulmonary bypass system 27 which includes a pump which may be a roller pump or a centrifugal pump. Venous cannula 50 may optionally include one or more balloons or other occlusion members thereon for occluding the vena cava to allow for total cardiopulmonary bypass, as disclosed in application Ser. No. 08/250,721, filed May 27, 1994, now U.S. Pat. No. 5,478,309, the complete disclosure of which is incorporated herein by reference for all purposes. Venous cannula 50 is connected at its proximal end to an extracorporeal cardiopulmonary bypass system of the type well-known to those of skill in the art, which removes carbon dioxide, oxygenates and filters the blood before returning it to the body through arterial perfusion cannula 20. Such cardiopulmonary bypass systems are described more fully in aforementioned U.S. Pat. No. 5,478,309, which has been incorporated herein by reference. With cardiopulmonary bypass established, occlusion member 24 is expanded to fully occlude the ascending aorta, thereby blocking blood flow therethrough. A cardioplegic fluid is then infused through cardioplegia/venting lumen 29 from which it flows into the ascending aorta distally of occluding member 24 and into the coronary arteries, perfusing the myocardium and arresting heart contractions. Surgery may then be performed on the still heart. Fluids may be vented periodically from the ascending aorta by applying negative pressure through cardioplegia/venting lumen 29 thereby decompressing the heart and maintaining a blood-free surgical field. When the surgery is complete, occlusion member 24 is contracted (deflated), allowing warm, oxygenated blood from arterial return lumen 25 to reach the coronary arteries. Heart contractions will then resume, and the patient is weaned from bypass.

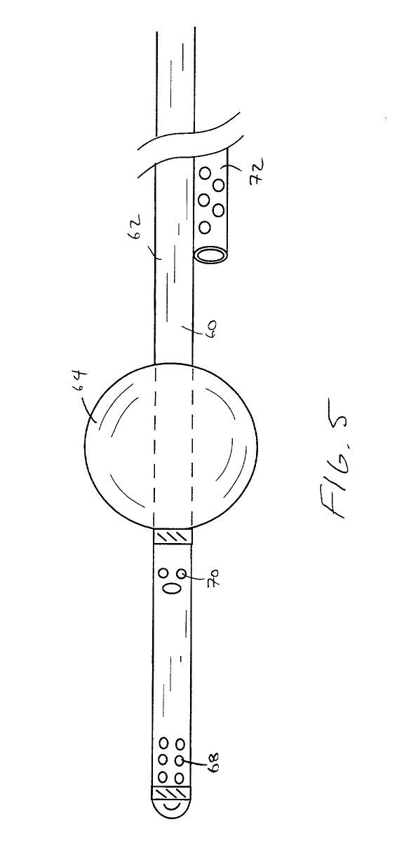

Further embodiments of the arterial perfusion cannula of the invention are illustrated in FIGS. 4-7. In these embodiments, arterial perfusion cannula 60 has a flexible shaft 62 and an occlusion member 64 attached at a location on the shaft suitable for placement in the ascending aorta from a peripheral artery such as the femoral or subclavian arteries or directly through the wall of the aorta. A distal extension 66 extends from shaft 62 distally of occlusion member 64 and is suitable for placement through the aortic valve into the left ventricle. Distal extension 66 may have a length of, for example, about 9 cm to reach into the left ventricle through the aortic valve when occlusion member 64 is expanded in the ascending aorta. A plurality of first openings 68 are disposed near the distal end of distal extension 66 for venting fluid from the left ventricle, and a plurality of second openings 70 are disposed in distal extension 66 just distally of occlusion member 64 (e.g. about 1-2 cm) for venting fluid from the ascending aorta and for infusion of cardioplegic fluid.

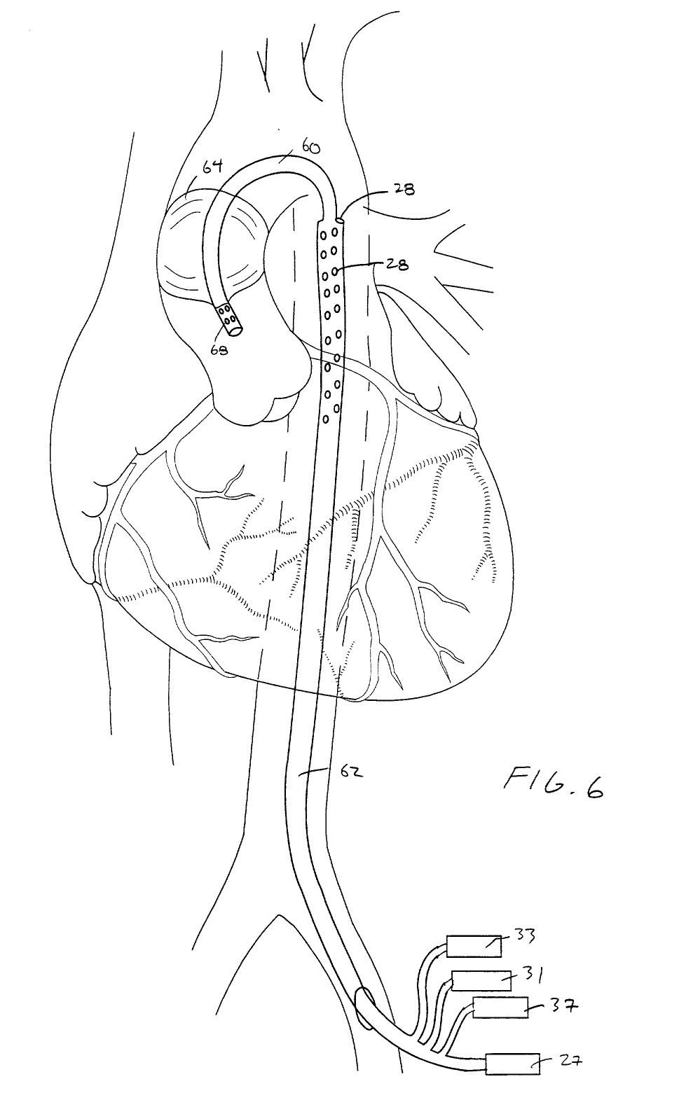

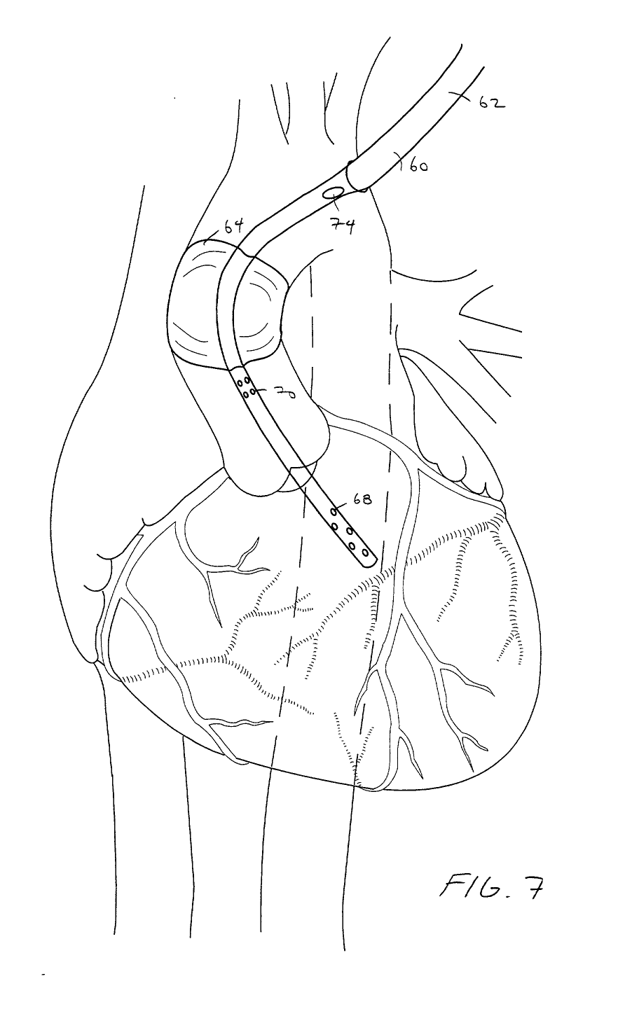

Shaft 62 includes an arterial return lumen 72 in communication with an arterial outlet 74 and having a proximal arterial return inlet 73 suitable for connection to the outlet of the cardiopulmonary bypass system 27. Arterial return lumen 72 and arterial outlet 74 are configured to deliver oxygenated blood to the patient at flows and pressures suitable for full cardiopulmonary bypass, thus having dimensions similar to those described above in connection with arterial perfusion cannula 20 of FIGS. 1-3. In the femoral embodiments described herein, the arterial return lumen 72 may have a shorter length so as to extend only into the femoral artery (FIG. 4), or may have a longer length sufficient to extend into the descending thoracic aorta (FIG. 6) or into the ascending aorta just proximally of occlusion member 64 (FIG. 5). When introduced into the subclavian artery, the arterial outlet 74 is positioned on shaft 62 so that it is located in the aortic arch when occlusion member 64 is in the ascending aorta. In any case, shaft 62 may be stepped-down or tapered to a smaller diameter distally of arterial outlet 74, thus giving the device minimal profile and maximum flexibility in its distal extremity. Additionally, as shown in FIG. 5, a plurality of sideholes may be provided along arterial return lumen 72 for enhanced blood flows.

Arterial perfusion cannula 60 further includes a ventricular venting lumen 75 in communication with first openings 68 and having a venting inlet 76 at its proximal end for connection to the suction source 33. A cardioplegia lumen 77 is in communication with second openings 70 and has a cardioplegia inlet 78 at its proximal end for connection to the source of cardioplegic fluid 31, and, if desired, to a suction source for venting the aortic root. Occlusion member 64 preferably is an inflatable balloon, and an inflation lumen 79 is in communication with the interior of occlusion member 64 and has a proximal inflation fluid inlet 80 for connection to the source of inflation fluid 37. Shaft 62 and occlusion member 64 may be of similar materials and construction as those of shaft 22 and occlusion member 24 described above in reference to FIGS. 1-3, except as otherwise described here.

Arterial perfusion cannula 60 preferably further includes one or more radio-opaque markers or bands 82 mounted to shaft 62 or to distal extension 66. Such bands facilitate fluoroscopic visualization of the device when utilized in a closed-chest operation. Referring to FIG. 4, the distal band 82 is preferably positioned about 2 cm from the distal end and the proximal band 82 is preferably positioned about 9 cm from distal end. The bands 82 help to locate the distal tip and the balloon when the cannula 60 is visualized fluroscopically.

While certain embodiments of the present invention relating to arterial perfusion cannulas and methods for providing cardiopulmonary bypass pump support during heart surgery have been described, it is to be understood that they are subject to many modifications without departing from the spirit and scope of the claims as recited herein.

What is claimed is:

1. A multichannel catheter having distal and proximal ends, comprising:
a first channel extending substantially the length of the catheter, the first channel comprising a major portion of an available channel volume of the catheter, the first channel being defined by the wall of the catheter, and being closed at its distal end;
a second channel extending substantially the length of the catheter parallel to said first channel but independent thereof, the second channel being integrated into the wall of the first channel and being open at its distal end;
a third channel extending substantially the length of said catheter parallel to said first and second channels but independent thereof, the third channel comprising, in combination with the second channel, a minor portion of the available channel volume of the catheter and being integrated into the wall of the first channel, the third channel having an opening and being spaced from the second channel;
a plurality of openings in the wall of the catheter near the distal end of said catheter and communicating only with said first channel; and
an inflatable means integrated into the distal end of the catheter between said first channel openings and said second channel distal opening and with the opening of said third channel in fluid communication with the interior of the inflatable means, wherein the catheter is of a size suitable for insertion into a blood vessel of mammal.

2. The catheter of claim 1, wherein:
the first channel comprises at least about ninety three percent of the available channel volume of the catheter; and
the third channel, in combination with the second channel, comprising not more than about seven percent of the available channel volume of the catheter.

3. The catheter of claim 1, wherein:
the first channel comprising at least about seventy percent of the available channel volume of the catheter; and
the third channel, in combination with the second channel, comprising not more than about thirty percent of the available channel volume of the catheter.

4. The catheter of claim 3 wherein the outflow capacity of the plurality of openings communicating with said first channel exceeds the inflow capacity into the first channel.

5. The catheter of claim 4 wherein a majority of the plurality of openings are elongate with the length of each elongate opening being parallel to the length of the catheter.

6. The catheter of claim 3 wherein the catheter is of a length that is sufficient to allow insertion into a femoral artery and positioning such that the distal end of the catheter is located in the ascending aorta such that the openings communicating with the first channel are positioned substantially adjacent the great arteries.

7. The catheter of claim 3 wherein the catheter is made using an extrusion technique.

8. The catheter of claim 3 wherein markings are positioned near the proximal end of the catheter to mark the distance from the distal end of the catheter.

9. A process of preparing a multichannel catheter that is of a size suitable for insertion into a blood vessel of a mammal, which process comprises:
extrusion molding a catheter having distal and proximal ends wherein the catheter comprises:
a first channel extending substantially the length of the catheter, the first channel comprising a major portion of the available channel volume of the catheter and being defined by the wall of the catheter;
a second channel extending substantially the length of the catheter parallel to said first channel but independent thereof, the second channel being integrated into the wall of the first channel;
a third channel extending substantially the length of said catheter parallel to said first and second channels but independent thereof, the third channel comprising, in combination with the second channel, not more than a minor portion of the available channel volume of the catheter, the third channel being integrated into the wall of the first channel and being spaced from said second channel.

10. The process of claim 9 wherein the extrusion molding step is carried out with the first channel being at least about ninety three percent of the available channel volume of the catheter

11. The process of claim 9 wherein the extrusion molding step is carried out with the first channel being at least about seventy percent of the available channel volume of the catheter, and the third channel, in combination with the second channel, being not more than about thirty percent of the available channel volume.

12. The process of claim 11 further comprising the steps of:
forming a plurality of openings in the wall of the catheter near the distal end of said catheter and communicating only with said first channel;
integrating an inflatable means into the distal end of the catheter positioned distal to said first channel openings so that the inflatable interior of the means is in fluid communication with said third channel through an opening in the wall of the catheter, and
forming at least one opening positioned distal to the inflatable means and communicating with said second channel; and
closing the distal end of said first channel.

13. The process of claim 11 wherein the outflow capacity of said plurality of openings communicating with said first channel exceeds the inflow capacity of the first channel.

14. The process of claim 11 wherein a majority of the plurality of openings are elongate with the length being parallel to the length of the catheter.

15. The process of claim 11 wherein the catheter is of a length that is sufficient to allow insertion into a femoral artery and positioning such that the distal end of the catheter may be located in the ascending aorta such that the openings communicating with the first channel are positioned substantially adjacent the great arteries.

16. A process for providing oxygen-rich blood to a patient's arterial circulation while providing a biologically active fluid to the heart of the subject, which process comprises:
positioning a multichannel catheter having a proximal end and a distal end in the patient's aorta, wherein said multichannel catheter comprises:
a first channel extending substantially the length of the catheter, the first channel comprising a major portion of the available channel volume of the catheter and being closed at the distal end of said catheter, the first channel being defined by the wall of the catheter;
a second channel extending substantially the length of the catheter parallel to said first channel but independent thereof and being integrated into the wall of the first channel;
a third channel extending substantially the length of said catheter parallel to said first and second channels but independent thereof, the third channel comprising, in combination with the second channel, a minor portion of the available channel volume of the catheter and being integrated into the wall of the first channel and spaced from said second channel;
a plurality of openings near the distal end of said catheter communication only with said first channel;
at least one opening at the distal end of the catheter communicating with said second channel;
an inflatable means integrated into the distal end of the catheter between said first channel openings and said second channel opening and communicating with said third channel through an opening in the wall of the catheter;
providing a source of oxygen-rich blood to the proximal end of said first channel;
providing a source of biologically active fluid to the proximal end of said second channel;
providing a source of fluid for inflating said inflatable means at the proximal end of said third channel;
positioning said multichannel catheter within the subject's blood circulatory system such that the distal end of said catheter is positioned in the ascending aorta so that the first channel openings are located upstream of the inflatable means, the inflatable means being located on the cephalid side of the aortic valve and the distal end of the second channel being located downstream of the inflatable means and proximate the aortic valve;
optionally inflating said inflatable means to block the flow of blood to the heart;
pumping biologically active fluid into the heart;
pumping oxygen-rich blood through said first channel out the first channel openings at a rate sufficient to maintain the subject's metabolism and perfusion;
optionally performing cardiovascular surgery on the heart as needed; and
maintaining the circulatory support for said subject as needed.

17. The process of claim 16 wherein the positioning step is carried out with the first channel comprising at least about ninety three percent of the available channel volume of the catheter.

18. The process of claim 16 wherein the positioning step is carried out with the first channel comprising at least about seventy percent of the available channel volume of the catheter, and the third channel, together with the second channel, being no more than about thirty percent of the available channel volume of the catheter.

19. The process of claim 18, wherein the biologically active fluid is a cardioplegia solution and the cardiovascular surgery is cardiac surgery.

20. A process for performing cardiovascular surgery, which process comprises:
inserting at least one cannula into the mammal's peripheral veins so that the distal open end of the cannula is adjacent the vena cava regions of the mammal's heart and the proximal end of the cannula is attached to a cardiopulmonary machine through a pump, said cardiopulmonary machine having a blood oxygenation means fluidly connected to said pump;
inserting a multichannel catheter having a proximal end and a distal end into a femoral artery, wherein said multichannel catheter comprises:
a first channel extending substantially the length of the catheter, the first channel comprising a major portion of the available channel volume of the catheter, the first channel being closed at the distal end of said catheter and being defined by the wall of the catheter;
a second channel extending substantially the length of the catheter parallel to said first channel but independent thereof and being integrated into the wall of the first channel;
a third channel extending substantially the length of said catheter parallel to said first and second channels but independent thereof, the third channel comprising, in combination with the second channel, a minor portion of the available channel volume of the catheter, the third channel being integrated into the wall of the first channel and spaced from said second channel;
a plurality of openings near the distal end of said catheter in communication only with said first channel;
at least one opening at the distal end of the catheter communicating with said second channel;
an inflatable means integrated into the distal end of the catheter between said first channel openings and said second channel opening and communicating with said third channel through an opening in the wall of the catheter;
positioning said multichannel catheter within the subject's blood circulatory system such that the distal end of said catheter is positioned in the ascending aorta such that the first channel openings are located upstream of the inflatable means and proximate the great arteries, the inflatable means being located on the cephalid side of the aortic valve and the distal end of the second channel is located downstream of the inflatable means and proximate the aortic valve;
providing a source of oxygenated blood from the cardiopulmonary machine to the proximal end of said first channel;
providing a source of biologically active fluid to the proximal end of said second channel;
providing a source of fluid for inflating said inflatable means to the proximal end of said third channel;
inflating said inflatable means to block the flow of blood to the heart;
optionally pumping cardioplegia solution into the heart to arrest the mammal's heart;
pumping oxygen-rich blood through the first channel out the first channel openings at rate sufficient to maintain the subject's metabolism and perfusion;
removing oxygen depleted blood from the mammal's vena cavae regions through the femoral vein cannula by applying a negative pressure using the centrifugal pump;
performing cardiovascular surgery as needed; and
maintaining the circulatory support for said subject as needed.

21. The process of claim 20 wherein the inserting step is carried out with the first channel comprising at least about ninety three percent of the available channel volume of the catheter.

22. The process of claim 20 wherein the inserting step is carried out with the first channel comprising at least about seventy percent of the available channel volume of the catheter, and the third channel, together with the second channel, being no more than about thirty percent of the available channel volume of the catheter.

23. A single multichannel catheter useful for extracorporeal circulation of blood to a patient undergoing cardiovascular surgery wherein the catheter comprises at least three independent channels and an expandable balloon at one end of the catheter,
a first, largest channel of a size to allow delivery of an amount of blood to the patient that is sufficient to support the patient metabolism and perfusion throughout the surgery,
a second channel, smaller than the first channel and integrated into the wall of the first channel, said second channel suitable for delivering cardioplegia solution to the heart and venting the left heart, and
a third channel also smaller than the first channel and integrated into the wall of the first channel, said third channel being suitable for delivery of a fluid to the balloon for its expansion when positioned in the ascending aorta to occlude the flow of blood.

24. The catheter of claim 23 of a length sufficient to be inserted throughout the femoral artery and positioned so that the balloon is positioned in the ascending aorta.

25. The catheter of claim 23 wherein the blood is delivered to the patient through openings in the wall of the first channel that are upstream of the balloon and the cardioplegia solution is delivered and the left heart is vented through an opening in the second channel that is downstream of the balloon.

26. In a method for performing cardiovascular surgery on a patient using a cardiopulmonary machine for extracorporeal circulation of blood, the improvement that comprises using a single, multichannel catheter for the extracorporeal circulation wherein the multichannel catheter comprises:
at least three independent channels and an expandable balloon at the distal end of the catheter;
a first, largest channel of a size to allow delivery of an amount of blood to the patient that is sufficient to support the patient metabolism and perfusion throughout the surgery;
a second channel, smaller than the first channel and integrated into the wall of the fist channel, said second channel suitable for delivering cardioplegia solution to the heart and venting the left heart; and
a third channel also smaller than the first channel and integrated into the wall of the first channel, said third channel being suitable for delivery of a fluid to the balloon for its expansion when positioned in the ascending aorta to occlude the flow of blood.

27. The method of claim 26 wherein the blood is delivered to the patient through openings in the wall of the first channel that are upstream of the balloon and the cardioplegia solution is delivered through the second channel out an opening that is downstream of the balloon.

28. The method of claim 27 wherein the surgery is open-chest surgery and the catheter is inserted through the patient's aorta or one of the great arteries and positioned so that the balloon is located in the ascending aorta to occlude the flow of blood to the heart

29. The method of claim 27 wherein the surgery is minimally invasive surgery and the catheter is inserted into the patient through the patient's femoral artery and positioned so that the balloon is located in the ascending aorta to occlude the flow of blood to the heart.

30. A method of delivering a biologically active agent to a subject in need thereof, which method comprises administering the agent using a single, multichannel catheter for the extracorporeal circulation wherein the multichannel catheter comprises:
at least three independent channels and an expandable balloon at the distal end of the catheter,
a first, largest channel of a size to allow delivery of an amount of blood to the patient that is sufficient to support the patient metabolism and perfusion throughout the surgery;
a second channel, smaller than the first channel and integrated into the wall of the first channel, said second channel being suitable for delivering cardioplegia solution to the heart and venting the left heart; and
a third channel also smaller than the first channel and integrated into the all of the first channels, said third channel being suitable for delivery of a fluid to the balloon for its expansion when positioned in the ascending aorta to occlude the flow of blood.

31. The method of claim 30 wherein the agent is a cardioplegia solution delivered through the second channel to the heart of a patient in need thereof.
